(19) United States
(12) Patent Application Publication
(10) Pub. No.: US 2020/0030462 A1
(43) Pub. Date: Jan. 30, 2020

(54) GREEN SYNTHESIS OF BIOCOMPATIBLE AND NEAR INFRARED ACTIVE EUGENATE (4-allyl-2-methoxyphenolate) CAPPED IRON OXIDE NANOPARTICLES FOR DEEP TISSUE IMAGING AND THERAPY

(71) Applicant: Indian Institute of Technology, Indore, Indore (IN)

(72) Inventors: Gupta SHARAD, Indore (IN); Kharey PRASHANT, Indore (IN)

(21) Appl. No.: 16/043,901

(22) Filed: Jul. 24, 2018

(51) Int. Cl. A61K 49/18; A61K 36/61; C01G 49/08

(52) U.S. Cl. CPC A61K 49/1833; B82Y 5/00; C01G 49/08; A61K 36/61

(57) ABSTRACT

NIR active, biocompatible and multifunctional (magnetic, photo-acoustic, and photo-thermal agent) E-capped iron oxide nanoparticles for deep tissue imaging and cancer therapy, are synthesized by a simple, rapid, non-toxic, low-cost and efficient completely green synthesis technique using extract made from the leaves of a medicinal aromatic plant, Pimenta dioica.

### TECHNICAL FIELD

The present disclosure relates to biocompatible iron oxide particles and their synthesis.

### BACKGROUND

Nanomaterials are used in various forms for different applications such as sensors in electronics, storage devices in semi-conductor industries, delivery vehicles for diagnostics and therapy applications. The synthesis protocols for different types of nanomaterials vary depending upon size, shape and morphology.

Among the variety of nanomaterials, magnetic nanomaterials are of particular interest for clinical research in disease diagnosis and therapy, specifically for cancer therapy. Early stage diagnosis of cancer enables better therapy and improves life expectancy of the patients. Nanotechnology based approaches offer early disease diagnosis of cancer.

Nanomaterials have exhibited a great potential in molecular and cellular imaging, for cancer screening, and cancer therapy. One of the nanomaterials used for biomedical applications are superparamagnetic iron oxide nanoparticles (SPIONPs). Super paramagnetic iron oxide nanoparticles exhibit magnetic behavior only in presence of external magnetic field.

Numerous chemical methods have been developed to optimize the synthesis superparamagnetic iron oxide nanoparticles, such as co-precipitation, micro emulsions, sol-gel synthesis and hydrothermal reactions. Additionally, these nanoparticles (NPs) can also be prepared by the other methods such as microwave assisted synthesis, combustion synthesis, carbon arc, electrochemical synthesis, laser pyrolysis techniques etc. The chemical methods are preferred due to their cost effectiveness and large scale production possibility. However, there are several drawbacks with these methods. The chemical method for synthesis are carried out at high temperatures and involve excessive amount of toxic chemical solvents. Further the chemicals methods also have the issues with chemical and physical stability of the synthesized nanoparticles.

Therefore, there exist a need to develop an environmentally friendly, optimized method to synthesize biocompatible iron oxide nanoparticles (IONPs) with a controlled size range and magnetic behavior for efficient applications in health care.

### SUMMARY

The main aspect of the present disclosure, relates to biocompatible, near-infrared active and magnetic eugenate (4-allyl-2-methoxyphenolate) capped Iron Oxide Nanoparticles (E-capped IONPs) and to the method of synthesizing the same using biomass derived from aromatic medicinal plant such as P. dioica for deep tissue imaging and cancer therapy, wherein a completely green synthesis technique is used to produce biocompatible, NIR active and multifunctional (magnetic, photo-acoustic and photo-thermal agent) E-capped IONPs comprising the steps of (a) preparing an aqueous solution of extract of leaves of aromatic plant, (b) preparing aqueous solution by dissolving Ferrous chloride tetrahydrate and Ferric chloride hexahydrate in 1:2 in 100 ml of deionized water, (c) heating the said solution prepared in step (b) for 15 minutes at a temperature of 60° C. by continuously stirring at 600 rpm, to obtain a yellowish colored solution. (d) adding 10 ml of the aqueous solution of aromatic plant extract drop by drop in aqueous solution of Ferrous chloride tetrahydrate and Ferric chloride hexahydrate; (e) wherein addition of plant extract turns the yellowish colour of the solution into reddish brown in colour; (f) adding 50 ml of 1M baking soda solution drop by drop to increase the pH of the solution to precipitate uniform magnetite nanoparticles. (g) Filtering and washing the magnetic nanoparticles obtained, thrice with deionized water followed by ethanol wash and then drying these E-capped IONPs magnetic nanoparticles.

### OBJECT OF THE PRESENT INVENTION

The principal object of the present invention is to produce a biocompatible, near-infrared active and multifunctional (magnetic, photo-acoustic and photo-thermal) eugenate (4-allyl-2-methoxyphenolate) capped iron oxide nanoparticles (E-capped IONPs).

Another object of the invention is to synthesize biocompatible, near-infrared active and magnetic eugenate (4-allyl-2-methoxyphenolate) capped iron oxide nanoparticles (E-capped IONPs) using a completely green chemistry approach.

Yet another object of the invention is to synthesize biocompatible, near-infrared active and magnetic eugenate (4-allyl-2-methoxyphenolate) capped iron oxide nanoparticles (E-capped IONPs) using biomass derived from aromatic medicinal plant such as P. dioica for deep tissue imaging and cancer therapy.

Yet another object of the invention is to synthesize biocompatible, near-infrared active and magnetic eugenate (4-allyl-2-methoxyphenolate) capped iron oxide nanoparticles (E-capped IONPs) using single step green synthesis technique that is simple and efficient.

Yet another object of the invention is to synthesize near-infrared active and magnetic eugenate (4-allyl-2-methoxyphenolate) capped iron oxide nanoparticles (E-capped IONPs) using single step green synthesis for biomedical applications.

### BRIEF DESCRIPTION OF THE DRAWINGS

The detailed description is described with reference to the accompanying figures.

FIG. 1, illustrates leaf of Pimenta dioica used to prepare the extract.

FIG. 2, illustrates step-wise synthesis of biocompatible eugenate (4-allyl-2-methoxyphenolate) capped iron oxide nanoparticles (E-capped IONPs) obtained by green synthesis.

FIG. 3, illustrates magnetic eugenate (4-allyl-2-methoxyphenolate) capped iron oxide nanoparticles (E-capped IONPs) obtained by green synthesis.

FIGS. 4a-4d illustrate characterizations of synthesized biocompatible eugenate (4-allyl-2-methoxyphenolate) capped iron oxide nanoparticles (E-capped IONPs).

FIG. 5, illustrates a plausible mechanism of formation of NIR active and magnetic eugenate (4-allyl-2-methoxyphenolate) capped iron oxide nanoparticles (E-capped IONPs) by interaction of functional group of eugenol present in P. dioica plant's leaf extract.

### DETAILED DESCRIPTION

The present invention relates to near-infrared active and magnetic eugenate (4-allyl-2-methoxyphenolate) capped Iron Oxide Nanoparticles (E-capped IONPs) for deep tissue imaging and therapy, and more particularly for the completely green synthesis of biocompatible, near-infrared active and multifunctional (magnetic, photo-acoustic and photo-thermal agent) E-capped Iron Oxide Nanoparticles using biomass derived from aromatic medicinal plant such as P. dioica for deep tissue imaging and cancer therapy.

The present invention discloses biocompatible, near-infrared active and magnetic E-capped IONPs for the biomedical applications. The present invention involves the method of making the same using natural resources. In this technique plant extract is used to synthesize E-capped IONPs. Completely green synthesized IONPs can be more suitable in healthcare fields in comparison to conventionally synthesized nanoparticles using chemical processes, which have toxic chemical species adsorbed on the surface of nanoparticles.

The present invention discloses biocompatible and multifunctional (magnetic, photo-acoustic, and photo-thermal agent) E-capped IONPs, synthesized using a simple, rapid, non-toxic, low-cost and efficient single step green synthesis technique, wherein the magnetic and NIR active E-capped IONPs are synthesized using abundantly available biomass derived from aromatic medicinal plant such as P. dioica

A green synthesis technique disclosed by the present invention uses plant extracts for the synthesis of nanoparticles. A number of phytochemicals such as alkaloids, flavonoids, phenols, terpenoids, quinines, tannins etc. are present in plants extract that influence the mechanism of nanoparticles synthesis. Plant extract has also been used as a reducing agent for the precursor ions and for preventing the mutual agglomeration of nanoparticles. In green synthesis process the rate of formation and yield of nanoparticles are affected by the choice of plant, nature and concentration of constituent phytochemicals present in plant extract. Also, the pH value, temperature and contact time play an important role in synthesis of these nanoparticles. However, green synthesis of metal nanoparticles using plants is currently under development. Green synthesized iron oxide nanoparticles can be more suitable in healthcare fields in comparison to conventionally synthesized nanoparticles using chemical processes, which have toxic chemical species adsorbed on the surface of nanoparticles. The present invention involves, NIR active and magnetic E-capped IONPs produced using a completely green approach. It is an ecofriendly, cost effective, pollution free, rapid synthesis technique. Green synthesized E-capped iron oxide nanoparticles are characterized using different conventional techniques.

Experimental analysis has been carried out and nanoparticles have been synthesized with NIR activity using green chemistry based technique, wherein a simple and efficient single step completely green synthesis technique has been performed to produce NIR active, biocompatible and multifunctional (magnetic, photo-acoustic, and photo-thermal agent) E-capped IONPs. The NIR active and magnetic E-capped IONPs are synthesized using abundantly available biomass derived from aromatic medicinal plants.

The present invention focuses to reduce the complexity, cost and toxicity issues of existing techniques to synthesize the nanoparticles for biomedical applications. These green synthesized magnetic E-capped IONPs will be used as exogenous contrast agent for multi-modal deep tissue imaging for early stage disease diagnosis and therapy.

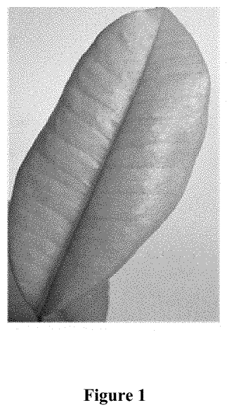

FIG. 1, shows leaf of Pimenta dioica, an aromatic to prepare the extract. The leaves of Pimenta dioica are washed with deionized water. Washed leaves are cut into small pieces and dried. Dried leaves are crushed with mortar and pestle followed by soaking 20 g in 100 ml of deionized water and heating for 5-10 minutes at 70-80° C. The resulting extract is filtered using Whatman No. 42 filter paper. The filtrate is collected in a clean, dried conical flask and stored at 4° C. for further use, so as to minimize possible contamination and also to maintain its bioactivity for a longer period of time.

Pimenta dioica (All spices) is an aromatic plant, belongs to the botanical spice-group of Pimenta Lindl. (“Allspice”), a Myrtaceae family. The essential oil extracted from Pimenta dioica have typical aroma of a combination of pepper, nutmeg, clove and cinnamon and hence named as All Spice. Pimenta dioica has been used for a variety of human endeavors, such as in perfumery industry, food spice, as a natural pesticide, and in folk medicine. In modern herbal medicine, Allspice extract has been used due to the abundance of phenolic compound Eugenol (4-allyl-2-methoxyphenol), which is a phenyl propene (an organic medicinal compound belongs to a class of phenylpropanoids found in plants) and several studies have reported its pharmacological activities. It composes 60-90% of the essential oil extracted from Allspice leaves.

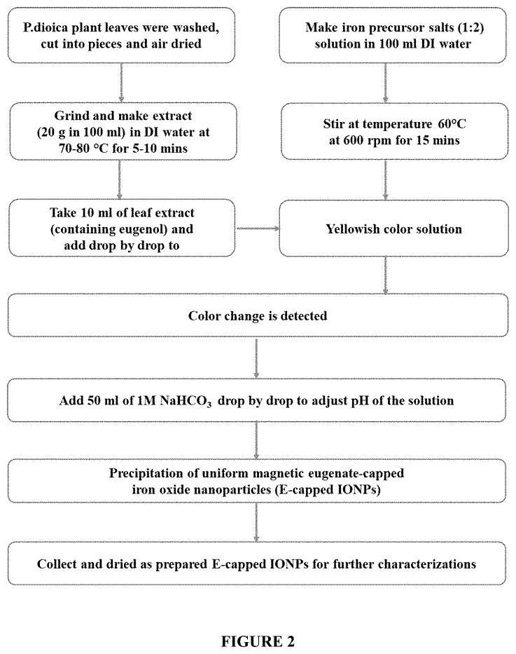

FIG. 2, illustrates, single step, completely green synthesis technique to obtain NIR active biocompatible and multifunctional (magnetic, photo-acoustic, and photo-thermal agent) E-capped IONPs.

FIG. 2, illustrates green synthesis, said green synthesis is a technique that reduces the use or generation of hazardous substances in the production and application of chemical products. The present invention focuses on plant-mediated synthesis of iron oxide nanoparticles (IONPs), a green chemistry approach that connects nanotechnology with plants. In the present invention, completely green synthesis procedure has been used wherein aromatic medicinal plant P. dioica and a FDA approved Sodium Hydrogen Carbonate (Baking Soda) are used for the formation of E-capped IONPs. So these completely green synthesized E-capped IONPs would be non-toxic in comparison to chemically synthesized IONPs which can show long term effect also, due to use of hazardous chemical that adsorbs on nanoparticles surface. Furthermore this completely green synthesis of E-capped IONPs is proposed as a cost-effective and environmentally friendly alternative to existing chemical and physical methods.

The present invention discloses an NIR active, eugenate (4-allyl-2-methoxyphenolate) capped Iron Oxide Nanoparticles (E-capped IONPs) with the completely green synthesis process of making the same. These E-capped IONPs showing different properties such as NIR activity, bio-compatibility and magnetic behavior. By virtue of such advantageous properties these completely green synthesized E-capped IONPs can be used for different biological and biomedical applications (in combination with above mentioned properties also). Since such properties incorporated in a completely Green synthesized E-capped IONPs. It can easily replace chemically synthesized IONPs used for biomedical applications.

### Experimental Details

Chemicals used for synthesis of NIR active IONPs are:
Ferrous chloride tetrahydrate (FeCl₂.4H₂O≥99.99%),
Ferric chloride hexahydrate (FeCl₃.6H₂O≥99.99%),

### Ethyl Alcohol (99.9%),

Sodium hydrogen carbonate (NaHCO₃, baking soda FDA approved).
Aqueous solutions of all these chemicals are prepared using deionized water.
The present invention uses leaves of Pimenta dioica, to prepare the extract as described in FIG. 1.

Preparation of Extract

Plant leaves were washed with deionized water. The leaves are then cut into small pieces and dried. Dried leaves were crushed with mortar and pestle followed by soaking 20 g in 100 ml of deionized water and heating for 5-10 minutes at 70-80° C. The resulting extract was filtered using Whatman No. 42 filter paper. The filtrate was collected in a clean, dried conical flask and stored at 4° C. for further use, so as to minimize possibility of contamination and maintain its bioactivity for long period

Green Synthesis of E-Capped Iron Oxide Nanoparticles

Ferrous chloride tetrahydrate (FeCl₂.4H₂O) and Ferric chloride hexahydrate (FeCl₃.6H₂O) were dissolved in 100 ml of deionized water in 1:2 ratio. This solution was heated at 60° C. for 15 minutes with continuous stirring at 600 rpm. Afterwards, 10 ml of the aqueous solution of aromatic plant leaf extract was added drop by drop. The addition of plant extract turned the yellowish colored solution into reddish brown colour. Then 50 ml of 1M Baking Soda solution was added drop by drop to increase the pH of the solution that allowed the precipitation of uniform magnetite nanoparticles and the colour changes from reddish brown to black. These nanoparticles formed were obtained by filtering and washing three times with deionized water followed by ethanol, wherein the nanoparticles were further washed and allowed to dry.

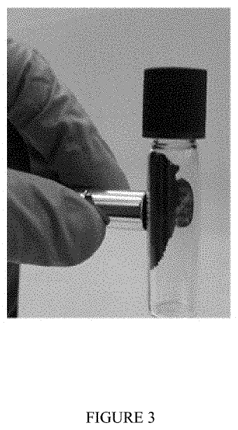

FIG. 3, illustrates iron oxide nanoparticles obtained by green synthesis, wherein nanoparticles obtained are attracted by an external magnet. FIG. 3 is showing the magnetic behavior of completely green synthesized E-capped IONPs in powder form. Magnetic behavior of these green synthesized nanoparticles is checked using a strong neodymium magnet in lab. Thus it is clear that completely green synthesized E-capped IONPs showing magnetic response (attracted by neodymium magnet) in external magnetic field.

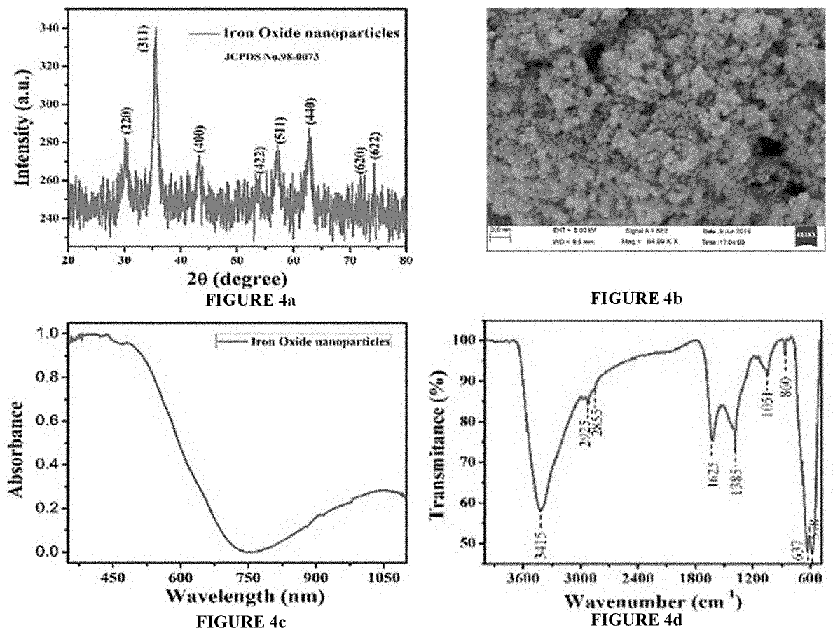

This is the first time green synthesized E-capped IONPs shows magnetic behavior with NIR absorbance by the use of FDA approved Sodium Hydrogen Carbonate (Baking Soda) only, instead of any hazardous chemical ex: ammonia, sodium hydroxide etc. By this completely green synthesis E-capped IONPs are formed with a phase showing magnetic behavior. (FIG. 3 & FIG. 4a).

FIGS. 4a-4d illustrate characterizations of synthesized E-capped biocompatible iron oxide nanoparticles. FIGS. 4a-4d show characterizations of completely green synthesized E-capped IONPs to understand the properties of the synthesized IONPs. FIGS. 4a-4d show Structural, Morphological, Optical etc. using different techniques.

FIG. 4a: shows the structural properties of completely green synthesized E-capped IONPs using X-ray diffraction technique. By using this technique the phase purity and crystallinity of synthesized E-capped IONPs is examined. The present invention identified different peaks at different diffraction angles which were then analyzed and well matched with the standard Magnetite (one of the magnetic phase of Iron Oxides) diffraction patterns from International center for data diffraction (JCPDS card no. 00-003-0045) indicating inverse cubic spinel crystal system. No characteristics peaks of impurities were observed, which confirms that the synthesized nanoparticles have high purity.

The average crystallite size was calculated using Debye-Scherrer equation,

$$d = \frac{κ λ}{β cos θ} .$$

The crystallite size was found to be 13.5 nm for magnetite nanoparticles. Furthermore it also indicates that the size of E-capped IONPs would be compatible for Bio medical applications.

FIG. 4b: shows the morphology, average particle size and shape of these completely green synthesized E-capped IONPs using a Field emission scanning electron microscope (FE-SEM). It was observed that nanoparticles are spherical in shape. The average particle size is less than about 15 nm. Size as estimated by the FESEM images is showing closer coincidence with their respective size calculation using XRD graphs (FIG. 4a). Moreover it shows that the morphology and size of E-capped IONPs would be compatible for Bio medical applications.

FIG. 4c: shows the Ultraviolet-Visible absorption (UV-Vis) of completely green synthesized E-capped IONPs. Maximum absorbance was observed at ˜435 nm, which corresponding to the formation of the iron oxide nanoparticles (IONPs). This figure also confirms that there is a significant absorption in the NIR wavelength range (750 nm-1050 nm) also. This corresponds to an inter valence charge transfer (IVCT) transition of Fe²⁺ to Fe³⁺ in octahedral sites of inverse spinel structured magnetite. So these completely green synthesized E-capped IONPs can be used for NIR imaging and photo thermal therapy in biomedical field.

Iron oxide nanoparticles don't show NIR activity without using any NIR dye or mixed with other inorganic or organic materials, with this completely green synthesis we have formed E-capped Iron Oxide nanoparticles with a phase, showing NIR absorption in these green synthesized E-capped IONPs. (FIG. 4c).

FIG. 4d: shows the FTIR spectra of completely green synthesized E-capped IONPs. By using FTIR spectroscopic technique, the functional group present in biomolecules of plant's biomass which are responsible for interaction (capping, reduction and stabilization) of biomolecules with Iron ions in green synthesis process were identified. FIG. 4d represents the major absorption bands of completely green synthesized E-capped iron oxide nanoparticles. The absorption peak at 3415 cm⁻¹ indicates the O—H (hydroxyl) group present. The peaks at 2925 and 2855 cm⁻¹ contribute to the C—H stretching vibration of the —CH₂ functional group. The absorption at 1625 cm⁻¹ represents the C═C aromatic stretching band of allyl group of eugenol. Absorption peaks at 1385 and 1051 cm⁻¹ correspond to the CH₃ bending and C—O stretching band of methoxy group of eugenol respectively. The peak at 860 cm⁻¹ reveals the existence of —CH bending band mode of benzene ring. Two significant peaks at 637 and 578 cm⁻¹ are associated with stretching vibration mode of Fe—O. The peaks in the region between 400-650 cm⁻¹ correspond to Fe₃O₄. Further it indicates that eugenol (60-90% of the essential oil extracted from Allspice leaves) present in P. dioica plant is responsible for green reduction reaction (not only reduction but capping and stabilizing also).

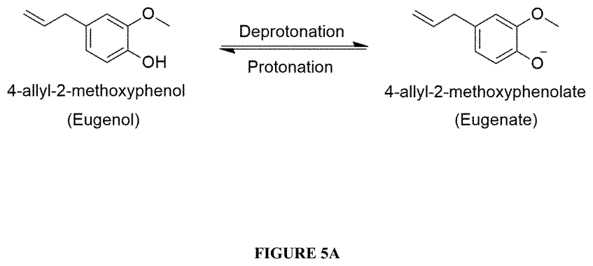

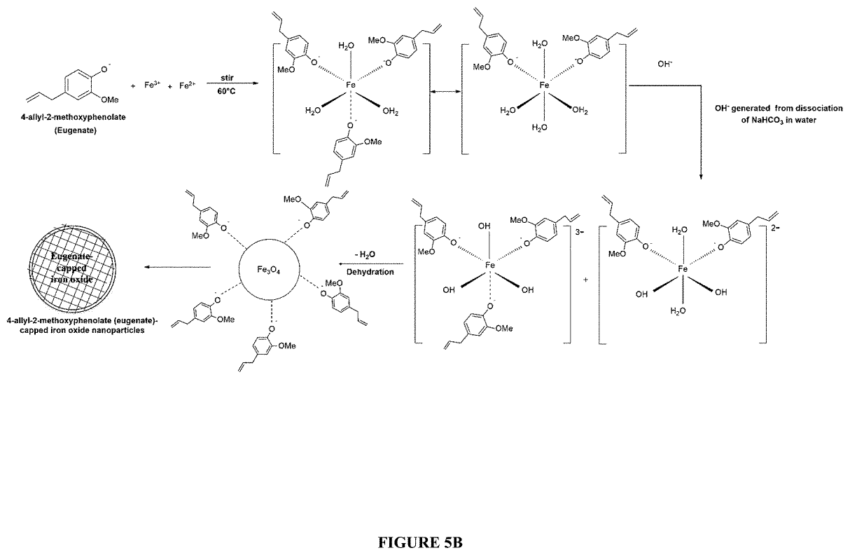

FIG. 5, illustrates the mechanism of formation of NIR active and magnetic E-capped iron oxide nanoparticles by interaction of functional group of eugenol present in P. dioica plant's leaf extract.

FIG. 5. Showing a plausible mechanism for interaction of eugenol (60-90% of P. dioica leaf extract) with iron metal ions and the formation of eugenate (4-allyl-2-methoxyphenolate) capped magnetic iron oxide nanoparticles (E-capped IONPs). Eugenol is slightly soluble in water, it carries a hydroxyl group on its phenolic ring. Eugenol can be protonated or deprotonated in aqueous solutions, as showed in FIG. 5a.

After deprotonation of eugenol in solution, the eugenate ion forms that interact with surface of Fe³⁺ and Fe²⁺ ions and form complex with iron ions. After adding baking soda (NaHCO₃) it gives OH⁻ ions to this reaction. Which then after form a ferric hydroxide, Fe(OH)₃ and ferrous hydroxide, Fe(OH)₂ capped with eugenate ions. Ferric hydroxide and ferrous hydroxide then dehydrated (—H₂O) and form Fe₃O₄, magnetite (magnetic iron oxide nanoparticles) capped and stabilized with eugenate, attached at Fe₃O₄ surface as shown in FIG. 5b.

FIG. 5b illustrates plausible mechanism of formation of eugenate (4-allyl-2-methoxyphenolate) capped magnetic iron oxide nanoparticles (E-capped IONPs) by eugenol present in P. dioica leaf extract.

In conclusion, the present invention discloses a product which is NIR active, biocompatible and multifunctional (magnetic, photo-acoustic, and photo-thermal agent) E-capped IONPs and produced by a simple, rapid, non-toxic, low-cost and efficient completely green synthesis technique, wherein the magnetic and NIR active E-capped IONPs are synthesized using extract made from the leaves of a medicinal aromatic plant, Pimenta dioica.

The above description along with the accompanying drawings is intended to describe the preferred embodiments of the invention in sufficient detail to enable those skilled in the art to practice the invention. The above description is intended to be illustrative and should not be interpreted as limiting the scope of the invention. Those skilled in the art to which the invention relates will appreciate that many variations of the described example implementations and other implementations exist within the scope of the claimed invention.

The following definitions and terms are provided for use in this disclosure and the appended claims that should be used unless otherwise specified. However, the invention is not intended to be limited to the specific terminology so selected.

As used herein, “nanoparticles” means particles with size in nanoscale range from about 1 to 100 nm diameter. Where nano is one billionth of a unit.

As used herein Pimenta dioica (P. dioica) or All spices is an aromatic plant, belongs to the botanical spice-group of Pimenta Lindl. (“Allspice”), a Myrtaceae family.

As used herein, a “plant leaf extract” include, ingredient of any natural organic chemical or combination of the chemicals found in a plant. As used herein, “plant leaf extract” also encompasses eugenol which is 60-90% of P. dioica plant leaf extract.

As used herein, “eugenate” refers to a deprotonated phenolic compound eugenol. “Eugenol” is a phenyl propene, which belongs to a class of phenylpropanoids.

As used herein “Biocompatibility’ is the ability of materials to interact with living system or tissue, without producing any adverse effects.

We claim:

1. A composition comprising: an iron oxide nanoparticles for deep tissue imaging and therapy wherein a surface of the nanoparticles capped with Eugenate (4-allyl-2-methoxyphenolate) to obtain Eugenate capped iron oxide nanoparticles.

2. The composition of claim 1, wherein the nanoparticles are biocompatible iron oxide nanoparticles.

3. The composition of claim 1, wherein the nanoparticles are near infrared active iron oxide nanoparticles.

4. The composition of claim 1, wherein the nanoparticles are magnetic iron oxide nanoparticle.

5. The composition of claim 1, wherein eugenate is deprotonated Eugenol (4-allyl-2-methoxyphenol), a phenolic compound.

6. The composition of claim 1, wherein phenolic compound Eugenol (4-allyl-2-methoxyphenol) is a phenyl propene.

7. The composition of claim 1, wherein phenyl propene is an organic compound belongs to a class of phenylpropanoids.

8. The composition of claim 1, wherein the nanoparticles have an average crystallite size 13.5 nm.

9. The composition of claim 1, wherein the nanoparticles are spherical in shape.

10. The composition of claim 1, wherein the average particle size is less than about 15 nm.

11. The composition of claim 1, wherein Ultraviolet Visible absorption of these biocompatible near-infrared active E-capped IONPs is observed at about 435 nm.

12. The composition of claim 1, wherein absorption in NIR wavelength range of these biocompatible near-infrared active E-capped IONPs is found to be from 750 nm to 1050 nm.

13. A process for making a nanoparticle composition, comprising:
a single step completely green synthesis technique to produce biocompatible NIR active and multifunctional eugenate (4-allyl-2-methoxyphenolate) capped iron oxide nanoparticles (E-capped IONPs) by preparing an aqueous solution of extract of leaves of an aromatic plant;
preparing an aqueous solution by dissolving a Ferrous chloride tetrahydrate and a Ferric chloride hexahydrate in 1:2 in 100 ml of deionized water;
heating the aqueous solution of Ferrous chloride tetrahydrate and a Ferric chloride hexahydrate for 15 minutes at a temperature of 60° C. by continuously stirring at 600 rpm, to obtain a yellowish colored solution;
adding 10 ml of the aqueous solution of the aromatic plant extract drop by drop in aqueous solution of Ferrous chloride tetrahydrate and Ferric chloride hexahydrate; wherein addition of plant extract turns the yellowish color of the solution into reddish brown in color;
adding 50 ml of 1M baking soda solution drop by drop to increase the pH of the solution to precipitate uniform magnetite nanoparticles; and
filtering and washing the magnetic nanoparticles obtained thrice with deionized water; followed by ethanol wash and then dried these magnetic nanoparticles.

14. The process as claimed in claim 13, wherein the extract of leaves of aromatic plant is prepared by;
washing the leaves and cutting them into small pieces;
drying the leaves;
crushing the dried leaves with mortar and pestle;
soaking the crushed leaves 20 g in 100 ml of deionized water;
heating the same for 5-10 minutes at a temperature of 70-80° C.; and
filtrating the extract obtained in a conical flask using whatman filter paper.

15. The process as claimed in claim 13, wherein the extract of leave of aromatic plant are selected from Pimenta dioica.

16. The process as claimed in claim 13, wherein the Eugenol (4-allyl-2-methoxyphenol) is 60-90% of P. dioica leaf extract.

17. The process as claimed in claim 13, wherein phenolic compound Eugenol (4-allyl-2-methoxyphenol) is a phenyl propene.

18. The process as claimed in claim 13, wherein phenyl propene is an organic compound belongs to a class of phenylpropanoids.

19. The process as claimed in claim 13, wherein the Eugenol acts as a reducing agent for green reduction reaction.

20. The process as claimed in claim 13, wherein the iron oxide nanoparticles surface capped with eugenate (4-allyl-2methoxyphenolate).

21. The process as claimed in claim 13, wherein eugenate is deprotonated Eugenol (4-allyl-2-methoxyphenol), a phenolic compound.

22. The process as claimed in claim 13, wherein baking soda is FDA approved Sodium hydrogen carbonate (NaHCO₃).

23. The process as claimed in claim 13, wherein aqueous solution of ferrous chloride tetrahydrate (FeCl₂.4H₂O) and ferric chloride hexahydrate (FeCl₃.6H₂O) are prepared by;
dissolving the ferrous chloride tetrahydrate and ferric chloride hexahydrate in 100 ml of deionized water; and
heating the solution for 15 minutes at a temperature of 60° C. by continuously stirring at 600 rpm, to obtain a yellowish colored solution.

24. The process as claimed in claim 13, wherein the Ferrous chloride tetrahydrate (FeCl₂.4H₂O) and Ferric chloride hexahydrate (FeCl₃.6H₂O) dissolved in 100 ml of deionized water are in ration of 1:2.

25. The process as claimed in claim 13, wherein plausible mechanism takes place by interaction of eugenol and iron metal ions to form eugenate (4-allyl-2-methoxyphenolate) capped magnetic iron oxide nano particles (E-capped IONPs).

26. The process as claimed in claim 13, wherein the biocompatible NIR active and multifunctional (E-capped IONPs) obtained can be used for diagnosis in molecular and cellular imaging.

27. The process as claimed in claim 13, wherein the biocompatible NIR active and multifunctional (E-capped IONPs) obtained can be used for diagnosis in cancer screening and cancer therapy.

28. The process as claimed in claim 13, wherein the biocompatible NIR active and multifunctional (E-capped IONPs) obtained can be used for diagnosis (MRI), treatment (Hyperthermia) and NIR activity such as Photothermal therapy and Photoacoustic Imaging.

29. The process as claimed in claim 13, wherein the biocompatible NIR active and multifunctional (E-capped IONPs) obtained can be used in Biological and Biomedical application.
